(19) United States
(12) Patent Application Publication
(10) Pub. No.: US 2002/0137999 A1
(43) Pub. Date: Sep. 26, 2002

(54) Medical alarm system

(76) Inventors: John J. Bandeian JR., Bristol, TN (US); John J. Bandeian III, Bristol, TN (US)

Correspondence Address: John J. Bandeian, Jr., 3169 W. State St., Bristol, TN, 37620, US

(21) Appl. No.: 10/150,982

(22) Filed: May 18, 2002

Related U.S. Application Data
(63) Continuation-in-part of application No. 09/636,376, filed on Aug. 11, 2000.

(51) Int. Cl. A61B 5/00

(52) U.S. Cl. 600/371; 340/604

(57) ABSTRACT

A monitoring and alarm system for detecting unexpected bleeding from a bleed site of a medical patient and for alerting medical attendant(s) thereof, wherein the system consists of an electrical circuit having a sensor for detecting the presence of blood at a bleed site, the sensor being operative to switch on a warning that such bleeding is occurring, and a structure for mounting the system proximate the bleed site.

This application is a continuation-in-part of applicants pending Ser. No. 09/636,376 filed Aug. 11, 2000 of same title.

### BACKGROUND OF THE INVENTION

1. Field

This invention concerns medical alarm systems and particularly systems which can quickly detect and warn the patient himself, the medical staff, or other attendants who may be caring for the patient, e.g., at home or in a hospital, of unexpected, i.e., inopportune, or excessive or heavy bleeding from a bleed site, either external or tubular as defined below. Such bleeding can occur, e.g., when a wound or incision reopens unexpectedly or when a tubular device such as an I.V. tube, syringe, catheter or the like is untimely or inadvertently pulled from or twisted or the like in a patients artery or vein, or when blood starts to back up in a tubular device which normally is supplying medicament to the patients blood stream.

2. Prior Art

Applicant is unaware of any prior systems or devices which are designed to function for the purposes as described above and which are relevant to the claims herein.

### SUMMARY OF THE INVENTION

The invention in one of its preferred embodiments is defined as a monitoring and alarm system for detecting such unexpected bleeding from a bleed site of a medical patient and for alerting medical attendant(s) thereof, wherein the system in its broad sense comprises electrical circuit means having sensing means for detecting the presence of blood adjacent a bleed site, said sensing means being operative to switch on a warning alarm that such bleeding is occurring, and means for mounting said system proximate said bleed site.

In another context the present system utilizes either or both of a first sensor means for external bleeding and a second sensor means for tubular bleeding.

The first sensor means in a first embodiment comprises a sheet-like blood porous base means of electrically non-conductive material having a proximal side adapted for mounting directly on a patients skin or on a blood porous protective gauze or bandage or the like thereon adjacent a bleed site, said base means having a distal side supporting a normally open electrical circuit means, electrically actuable alarm means electrically connected into said circuit means, and electrical contact switching means in said circuit means responsive to contact with blood to actuate said alarm means to alert said medical attendant(s) to said external bleeding.

The first sensor means in a second embodiment comprises a base means adapted for attachment in a desired position on a patients body over a potential bleed site, a wick means of light transmitting material mounted on said base means distally of said site and adapted to wick blood from said site, photoelectric switch means positioned on said base means for detecting a change in light transmittance thru said wick means as the result of blood wicked thereinto, and electrical circuit means comprising an alarm system associated with said switch means for generating an alarm that bleeding from said site is occurring.

The second sensor means in a preferred embodiment comprises photoelectric switch means having base means for mounting said switch means adjacent to a fluid transport tube for detecting a change in light transmittance thru said tube as the result of blood present therein, and electrical circuit means comprising an alarm system associated with said switch means for generating an alarm that blood is present in said tube.

### BRIEF DESCRIPTION OF THE DRAWINGS

The invention will be further understood from the drawings and description herein, wherein structures are enlarged for purposes of clarity and are not drawn in proportionate dimensions:

FIG. 1 is a cross-sectional view of one preferred embodiment of the present system positioned on a patients arm over the site of a wound or incision;

FIG. 2 is a view as in FIG. 1 showing a variation in the electrical circuit mounting;

FIG. 3 is a view of one exemplary type of electrical circuit useful in the present invention taken along line 3-3 of FIG. 1 in the direction of the arrows with only a representative number of pores shown in the base means;

FIG. 4 is a cross-sectional view taken along line 4-4 of FIG. 1 in the direction of the arrows and showing a wrap means for attaching the present system to a patients limb or body;

FIG. 5 is a top view of a special shape for the present system;

FIG. 6 is a view as in FIG. 1 but employing a wick and photoelectric switch rather than an electrical circuit such as 22;

FIG. 7 is a view as in FIG. 6 but employing a variation of the wick;

FIG. 8 is a view as in FIG. 1 but showing embodiment 8 employed in concert with a second sensor means having a photoelectric switch device;

FIG. 9 is a view as in FIG. 7 but showing embodiment 19 employed in concert with 27;

FIG. 10 is a top view of the combination of sensors of FIG. 9;

FIG. 11 is an enlarged view of the tube clamping element of FIG. 10; and

FIG. 12 is a schematic of an electrical alarm circuit for use with the photoelectric switch in the present invention.

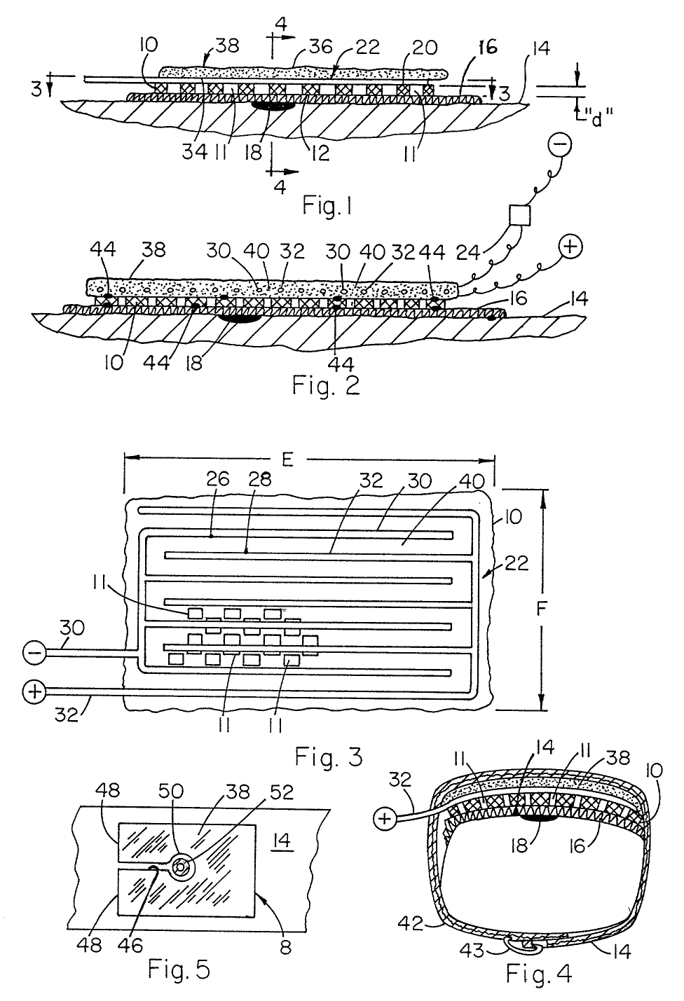

### DETAILED DESCRIPTION OF PREFERRED EMBODIMENTS

Referring to the drawings and with particular reference to the claims hereof, the invention in its broad embodiment comprises electrical circuit means 22, 41 having sensing means 8, 19, 27 for detecting the presence of blood adjacent a bleed site, said sensing means being operative to switch on a warning alarm means 24, 45 that such bleeding is occurring, and means 10, 21, for mounting said system proximate said bleed site 18.

In the context the more limited claims, the first embodiment of said first sensor means is generally designated 8 and comprises a relatively thick sheet-like base means 10 of electrically non-conductive, blood porous material having a proximal side 12 adapted for mounting directly on a patients skin 14 or on a porous protective gauze, bandage or other such porous medical item 16 thereon at a site 18 on the patients body where bleeding can occur, i.e., bleed site, said base means having a distal side 20 with a predesigned distance “d” from item 16, electrically actuable visual, sound or physical alarm means 24 electrically connected into circuit means 22, and electrical switching means comprising any adjacent electrical contact portions anywhere along electrical conductors 30 and 32 such as 26,28 in said circuit means responsive to bridging contact with electrolytic blood to close said circuit means and actuate said alarm means to alert medical attendant(s) to said excessive bleeding.

The base 10 must be of electrically non-conductive material such as polyolefins, PVC, polyester, urethanes, natural or synthetic rubbers or elastomers, any of which may be plastisols or which preferably are of foamed cellular structure to provide substantial flexibility and softness to the base. The base is preferably configured as a flexible netting type structure having a thickness of from about 1/32 to about 3/16 in., and having from about 6 to about 20 pores 11 per inch, most preferably from about 9 to about 16 pores per square inch, and wherein said pores average in area from about 0.002 to about 0.02 in², most preferably from about 0.005 to about 0.01 in². The pores can be of any shape including square, rectangular, round or oval. The manufactured dimensions E and F of the system is determined by the area to be monitored and can range, for example, from a square inch to 50 or more square inches.

The essential character of the base is that the pores must allow free flow of blood from the bleed site 18 to bridge the gap between the conductors 30,32 of the circuit 22. The sensitivity of the system to bleeding can, of course, be increased by reducing the values of “d”, e.g., by reducing the thickness of the base. This base thickness, while having an allowable wide dimensional range, should not be so thin as to allow normal or expected fluid leakage from the site to easily fill up the pores and bridge the circuit conductors, whether an intermediate barrier such as gauze 16 is used or not.

It is noted that the present system, preferably, is to be placed on the patient such that the blood would have to flow or migrate substantially upwardly against the force of gravity to reach the sensing means. Such placement more easily guarantees that casual seepage of blood or other body fluid from site 18 would not trigger the alarm, particularly where an intermediate absorptive element such as 16 is employed.

The electrical circuit 22 in a simple but very efficient form as shown in FIG. 4 consists of side by side conductor 30,32 which may be supported by and held directly on the distal side 20 of the base. These conductors may also be supported on either the proximal 34 or distal side 36 of a support such as 38, or encased by said support. This support, preferably, is highly absorptive of blood and constructed to allow rapid migration of blood throughout the support for bridging a gap such as 40 between the conductors.

The spacing of the conductors should be sufficiently small to allow blood to readily complete the circuit. Spacings of from about 0.1 to about 0.75 in., have been found to operate with great rapidity for a more than small or casual blood flow. In this regard, it is apparent that extremely sensitive switch means employing relays or the like in the circuitry can be employed to operate the alarm on very low electrical sensing or switching current flowing between the conductors. The use of low voltage battery sensing or switching power, e.g., 1-9 volts, is preferably, and, of course, can be relayed into house or hospital wiring, if desired.

The type of alarm is readily selected by one skilled in the art and can be, for example, flashing lights, ringing bell, speaker, or even the physical jostling of a chair or the like in which an attendant may be sitting, whether in or out of a hospital setting. An effective sound alarm is the Model BWDHWA WATCHDOG WATER Alarm marketed by Glentronics, Inc., Glenview, Ill.

Referring to FIG. 4, a wrap means 42 such as an Ace bandage and securement clip 43 or the like may be employed to hold the system to the patients arm, leg, or even torso or other body part. It is noted that the present system, particularly in embodiments where the various elements such as 16, 10, 22 and 38 are pre-assembled as a unit, can be placed in any exterior location on the body such as adjacent any body orifice and operate effectively to detect and alarm for excessive bleeding. In this regard, such a unit can be provided with adhesive material, preferably water insoluble and of adhesive bandage type, around the edges of the base or gauze for convenient attachment to the skin such as at locations 44.

Referring to FIG. 5 the present system 8 is configured with a slot or split 46 which extends from one edge 48 to an inner aperture such as 50 which passes completely thru the system from top to bottom. This aperture can be tailored in size and shape to accommodate a medical item 52 such as an I.V. tube, catheter or the like which is affixed, e.g., into a patients vascular system.

Referring to FIG. 6, the second embodiment of said first sensor means is generally designated 19 and comprises a base means 21, a wick means 13 and a photoelectric switch means comprising a light source (emission means) 15 and a light receiver 17. An electrical circuit means 23 having an alarm system such as 45 is connected into said switch means and an exemplary switch means, electrical circuit and alarm are shown schematically in FIG. 12.

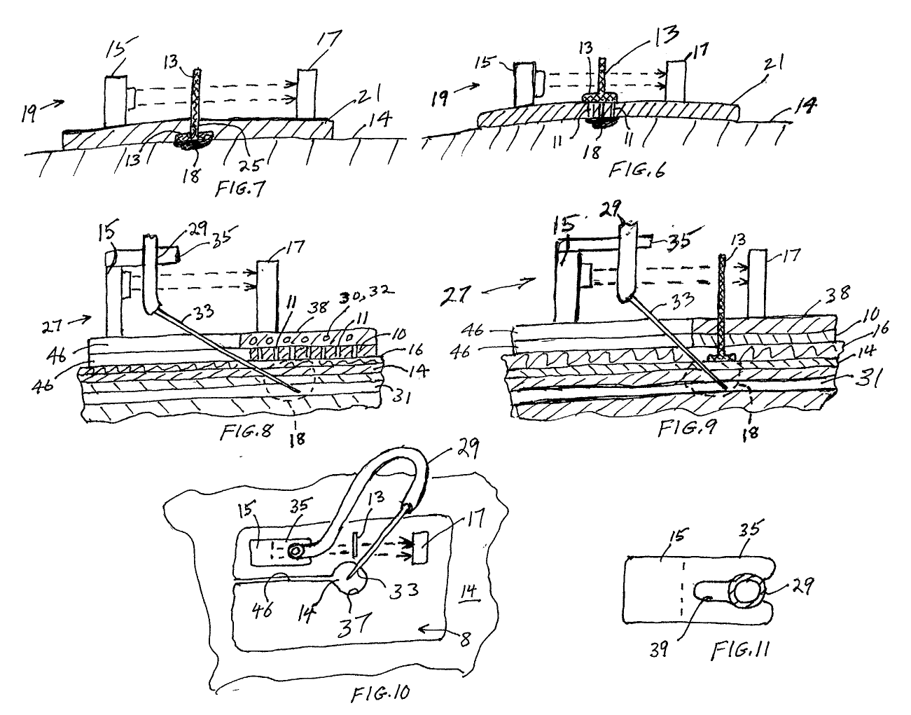

The base means 21 may be constructed of any material such as PVC which is suitable for medicinal use and which can support the wick means and photoelectric switch means. The base means preferably is used with a bandage such as 16 shown in FIG. 1 and may be provided with pores such as 11 which would allow the wick 13 to be affixed to the distal side of 21. Alternatively, as shown in FIG. 7, wick 13 can be supported by insertion thru and securement in an aperture such as 25 thru base 21. The wick may, of course, be any cross sectional configuration, preferably quite thin and rectangular to allow significant light transmittance therethrough and is made of any material such as felt cellulose paper, cotton or the like which will readily wick blood and which preferably is sufficiently rigid to be self-supporting. Where the wick is of thin material it can be supported in a wire frame or the like where the material is not self supporting.

The photoelectric switch relies on a decrease in light transmission thru a wick or fluid transport tube to trigger the bleeding alarm. For example, it is preferred that a decrease in light transmission of about 10% or more from normal be sufficient to trigger the alarm. It is noted that where medicament which has light blocking capacity is being infused thru an I.V. tube or the like, the sensitivity of the photoelectric switch can be adjusted to ensure that any triggering light blockage results only from actual external or tubular bleeding. A light source and a photoelectric cell are mounted on top of a base means and a wick material is mounted between the light source and the photoelectric cell. The light source could be an incandescent bulb or a light emitting diode. The light source, the wick material and the photoelectric cell are adjusted so that enough light reaches the cell to generate an electric current. The electric current drives a relay, which is normally closed. The relay is wired in series with a battery powered alarm. When the light reaching the photoelectric cell is diminished or ceases entirely, the relay opens and the alarm goes off If even a small amount of blood reaches the wick it will seep up the wick and decrease the light reaching the photoelectric cell which will trigger the alarm. This trigger mechanism can be used on a wound with an exposed artery, or it can be applied (minus the wick) for example, to the intravenous tubing in a large sized vein. If blood starts backing up the tubing because of reverse flow, the photoelectric cell will stop generating electricity and the alarm will sound.

The preferred photoelectric switch with the wick 13 and/or tube 29, and the alarm circuit with battery is a miniaturized unit which generates and sends out a signal to energize the control coil or solid-state circuitry of an industrial or process control device. The switch is activated when a beam of light is interrupted or changes intensity due to blood in its path. Such a unit is readily constructed to occupy, e.g., a space of about two cubic inches or less.

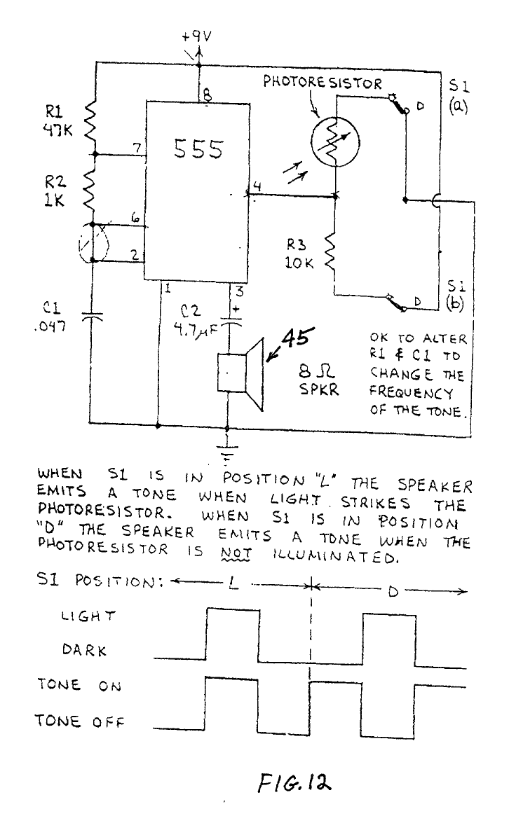

The second sensor means 27 for tubular bleeding is shown in FIGS. 8, 9 and 10 wherein for exemplary purposes the external bleeding sensor means 8 of FIGS. 1 and 5 are shown in combination with 27. Sensor 27 may, of course, be employed by itself, however, the combination with a sensor such as 8 provides for greatly enhanced patient safety.

Referring to FIGS. 8-11, a fluid transport tube such as 29 of light transmitting material such as clear PVC or the like plastic tubing as used, for example, for the infusion of various fluids into a patients vascular system, is connected to a syringe needle 33 as shown inserted into a vein 31. The tubing 29 is preferably supported on base 10 or support 38 in a generally upright posture by, for example, a clamping element 35 such as is shown in FIG. 11 attached to the component 15 of the photoelectric switch.

As depicted in FIGS. 5 and 10, the tube 29 and needle 33 are first positioned such that slot 46 of the sensor means 8 can be slid past the needle and the tube then clamped in position by element 35. For this purpose the photoelectric switch components and the wick, if present, may be positioned as shown in FIG. 10 for convenience of assembly. It is noted that as an alternative to slot 46, the opening 37 may be suitably enlarged to provide for insertion of the needle.

Referring to FIG. 11, the clamping element 35 is of sufficiently flexible material to allow a tube 29 to be pushed inwardly into slot 39 and held upright therein without significantly deforming or compressing the tube and restricting fluid flow therethrough.

It is noted that in the embodiment of the present invention where a wick is employed, the base, e.g., 10 or 21 preferably is constructed to allow the passage of blood only to the area of the wick as shown in FIGS. 6 and 7.

In all of the embodiments shown, the base means such as 10 or 21 can be readily modified to properly support the various sensor components and fluid transport tubes and the like.

This invention has been described in detail with particular reference to preferred embodiments thereof, but it will be understood that variations and modifications will be effected within the spirit and scope of the invention.

I claim:

1. A system for monitoring potential bleed sites of medical patients comprising electrical circuit means having sensing means for detecting the presence of blood adjacent a bleed site, said sensing means being operative to electrically switch on a warning alarm that such bleeding is occurring, and means for mounting said system proximate said bleed site.

2. The system of claim 1 wherein said potential bleed site is external or tubular.

3. The system of claim 1 wherein said sensing means is selected from the group consisting of (a) a normally open electrical circuit which is closed thru the bridging of electrical contacts by electrolytic blood, and (b) a normally closed photoelectrical circuit which is opened by the presence of blood within the light beam of said circuit.

4. The system of claim 3 wherein in the sensing means (b), a wick is present within said light beam, wherein said wick normally transmits a high percentage of said beam, and wherein with blood present in said wick, said beam is blocked sufficiently to open said circuit and activate said alarm.

5. The system of claim 3 wherein in the sensing means (b), a fluid transport tube is present within said light beam, wherein said tube normally transmits a high percentage of said beam, and wherein with blood present in said tube, said beam is blocked sufficiently to open said circuit and activate said alarm.

6. The combination structure of a monitoring and alarm first system for detecting excessive external bleeding from medical patients, and a monitoring and alarm second system for detecting tubular bleeding from medical patients, and for alerting medical attendant(s) of any such external or tubular bleeding, said first system comprising a relatively thick sheet-like base means of electrically non-conductive, blood porous material having a proximal side adapted for mounting directly on a patients skin or on a porous protective gauze or bandage or the like thereon at a site on the patients body where heavy bleeding can occur due to inadvertent opening of a wound or incision or due to inadvertent twisting, bending or extraction of a syringe, I.V. tube, catheter or, said base means having a distal side, a normally open electrical circuit means positioned adjacent said distal side and spaced from said proximal side a predesigned distance, electrically actuable visual, sound or physical alarm means electrically connected into said circuit means, and electrical switching means for said circuit means responsive to contact with blood to close said circuit means and actuate said alarm means to alert said medical attendant(s) to said excessive bleeding, wherein said second system comprises a photoelectric switch mounted on said combination structure and having light emission means and light receiving means, clamping means on said combination structure for supporting a fluid carrying light transmitting tube in a substantially vertical position between said light emission means and said light receiving means, said tube having one end adapted for connection to a syringe needle to be inserted into a patients blood vessel, whereby with the needle thus inserted, any blood backing up in said tube will change the light transmission thru the tube and generate an alarm signal.

7. The combination structure of claim 6 wherein said circuit means of said first system comprises at least two spaced apart electrical conductors positioned adjacent said distal side of said base means, wherein one conductor is connected to a power lead and the other conductor is connected to a ground lead of a power source, and wherein said switching means comprises at least one gap between contact portions of said spaced conductors and adapted to be bridged by blood to electrically connect said contact portions.

8. The system of claim 7 wherein said power source is selected from the group selected from battery power, house, clinic or hospital power, or generator power.

9. The system of claim 7 wherein said conductors are mounted on or imbedded in a non-conductive support means having a capacity for blood absorption and migration.

10. The system of claim 9 wherein said support means is affixed to said distal side of said base means, and wherein said base means is comprised of a flexible netting type structure having a thickness of from about 1/32 to about 3/16 in., and having from about 6 to about 20 pores per square inch, wherein said pores average in area from about 0.002 to about 0.02

11. The system of claim 10 wherein the material of said netting is comprised substantially of a material of the group consisting of polyolefin, PVC, polyester, polyurethane or natural or synthetic rubber or elastomers.

12. The system of claim 9 wherein the material of said support means comprises a mat of natural fibers.

13. The system of claim 9 wherein the material of said support means comprises a mat of felt.

14. The system of claim 9 wherein the material of said support means comprises a mat of blood absorptive paper.

15. The system of claim 9 wherein the material of said support means comprises a mat of blotter paper.

16. The system of claim 9 wherein said support means is physically attached to said base means by electrically non-conductive connector means.

17. The system of claim 1 wherein the material of said base means is substantially blood non-absorptive.

18. The system of claim 16 wherein wrap means is provided on said support means for making quick and stable attachment of said system to a patients body.

19. The system of claim 18 wherein the material of said base means is substantially blood non-absorptive.

20. The system of claim 16 wherein a layer of blood absorbing medical gauze is affixed to the proximal side of said base means to thereby provide a unitary bandaging and bleeding alarm unit.
